(19) United States
(12) Patent Application Publication
(10) Pub. No.: US 2025/0372209 A1
(43) Pub. Date: Dec. 4, 2025

(54) SYSTEMS AND METHODS FOR IDENTIFYING DNA SEQUENCES REGULATING PATTERN OF EXPRESSION FOR GENES OF INTEREST

(71) Applicant: NUTECH VENTURES, Lincoln, NE (US)

(72) Inventors: Michael C. Tross, Lincoln, NE (US); James C. Schnable, Lincoln, NE (US)

(21) Appl. No.: 19/226,059

(22) Filed: Jun. 2, 2025

Related U.S. Application Data
(60) Provisional application No. 63/654,690, filed on May 31, 2024.

(51) Int. Cl. G16B 40/00; C12N 9/22; C12N 15/11; C12N 15/82

(52) U.S. Cl. CPC G16B 40/00; C12N 9/226; C12N 15/111; C12N 15/8213; C12N 2310/20

(57) ABSTRACT

Systems and methods are presented for constructing, training, and utilizing a large contextual gene sequence model that can be provided with variable-length DNA sequence data from larger genomic intervals surrounding annotated genes to predict relative expression across a set of transcriptionally diverse tissues. Additionally, per-nucleotide saliency scores can be extracted from the large contextual gene sequence model, indicating which regions of the DNA sequence surrounding a target gene are associated with regulation of expression of the target gene in various tissue types of an organism. The model described herein surprisingly tolerated averaging across a given embedding of a DNA sequence, and the use of averaging across each embedding allowed the development of a transformer-based model capable of producing a constant set of outputs, indicating the relative expression across a fixed set of tissues, from variable lengths of DNA sequence.

### CROSS-REFERENCE TO RELATED APPLICATIONS

This application claims the benefit of and priority to U.S. Provisional Application No. 63/654,690, filed on May 31, 2024, which is incorporated by reference herein in its entirety.

This invention was made with government support under grant nos. 2020-68013-32371 and 2021-67021-35329 awarded by the U.S. Department of Agriculture (USDA) National Institute of Food and Agriculture (NIFA). The government has certain rights in the invention.

### TECHNICAL FIELD

The present disclosure generally relates to systems and methods for constructing, training, and utilizing artificial intelligence (AI) models for genetic research and engineering applications. More specifically, the present disclosure relates to systems and methods for constructing, training, and utilizing a large contextual gene sequence model to predict, from a DNA sequence of an organism, a pattern of mRNA and/or protein expression across different types of tissues of the organism, and to assess which regions of the DNA sequence are associated with regulation of the mRNA and/or protein expression across the different types of tissue of the organism.

### BACKGROUND

The evolution of species has historically relied on capitalization of natural genetic variation within their populations. With the advent of modern gene engineering technologies, such as CRISPR/Cas9, many researchers have explored the potential of genetic enhancement via targeted genome editing. Although large changes in gene expression can be achieved by the disruption of coding sequence via editing, adaptation has been largely driven by variation in non-coding regulatory sequence. Yet, the regulatory landscape of many species has not been fully explored.

The potential of gene editing to improve rates of genetic gain and engineer more resilient, resource-use-efficient, and nutritious crops is substantial. However, achieving the full potential of gene editing will require fine-tuning levels and gene expression patterns and not merely disrupting coding sequences, which is the most common editing approach used today in many crop and model species. While the identification of protein-coding sequences within genomes and, to some extent, the prediction of which sequence changes will have greater or smaller impacts on protein function have become increasingly straightforward tasks, the identification of the noncoding regulatory regions that determine where, when, and in what quantities protein-coding DNA sequences are transcribed into mRNA, as well as the prediction of how changes in these regulatory sequences will influence function, has remained far more challenging.

At least two distinct approaches to the identification of noncoding regulatory sequences have previously been used and validated on genome-wide scales: the identification of conserved noncoding sequences, and the identification of open chromatin regions. Comparison of orthologous genomic regions across related species can identify noncoding regions that, like many exons, exhibit slower rates of nucleotide sequence evolution, indicating that these regions are likely to be functionally constrained. Several of these functionally constrained noncoding sequences (i.e., conserved noncoding sequences) have been shown to function in regulating the expression of nearby genes. However, by definition, evolutionarily young regulatory regions will not be identified as conserved noncoding sequences. In addition, the smallest sequences that can be confidently identified as showing functional constraint in plant genomes are substantially larger than known transcription factor binding sites. Conserved noncoding sequences thus mark a subset of the functional regulatory sequences present in a given plant genome. Open-chromatin regions identified via a range of sequencing-based methods (e.g., Assay for Transposase-Accessible Chromatin using sequencing (ATAC-seq) and micrococcal nuclease digestion with deep sequencing (MNase-seq)) have been shown to contain a large majority of the genetic markers outside of gene bodies that are linked to variation in plant phenotypes. However, current open-chromatin methods tend to identify larger genomic intervals and likely represent a superset of regulatory sequences in plant genomes.

### SUMMARY

Over the past five years, a range of increasingly complex machine learning algorithms have been employed for the task of predicting gene expression—defined in various ways—from the nucleotide sequence. These models have demonstrated the ability to predict which of a pair of genes will be more expressed using DNA sequence starting one kilobase of the annotated transcription start site and extending one kilobase downstream of the transcription end site (in maize) and to predict which genes will exhibit differential expression in response to an external stress across maize and related species. Efforts to predict tissue-specific patterns of expression from DNA sequence have been less successful. A Bidirectional Encoder Representations from Transformers (BERT)-based transformer model exceeded controls, but only achieved low prediction accuracies (e.g., R²=0.092-0.192) in predicting absolute expression levels in individual maize tissues. Another transformer-based effort was able to accurately estimate the overall strength of individual promoter elements, but the technique exhibited poor performance in predicting differences in expression of the same genes across different tissues from proximal promoter sequences. Applicant recognized that long range models are needed to predict gene expression patterns from DNA sequence and appreciated that these models can also be used to understand which nucleotides play important roles in regulating gene expression and how DNA sequence changes may alter gene expression levels.

The embodiments described herein involve the training of an artificial intelligence (AI) model, a transformer-based machine learning model, to predict a pattern of differential gene expression across various tissues of an organism. Utilizing a gradient-based interpretation of the model, unique regulatory regions can be extracted from individual genes. The model has been validated using comparisons to two other existing approaches, including (i) a first approach known to identify only a subset of regulatory sequences for only a subset of genes, as well as (ii) a second approach that requires expensive and time-consuming molecular biology steps rather than occurring entirely in silico (as described for certain embodiments presented herein). The methods described herein address a key need among current biotechnology companies that have built out extensive pipelines for applying gene editing (e.g., CRISPR/Cas9) to support molecular farming, crop improvement, and synthetic biology efforts but are currently bottlenecked in their efforts by the challenges in engineering changes in the expression levels and expression patterns of native genes. A non-limiting example application described herein relates to the use of such model-based gradient approaches in uncovering the regulatory sequence in maize (Zea mays) and the transferability across species. For this example, saliency of the input DNA sequence in predicting relative tissue expression revealed peaks that localize in conserved non-coding sequence and open chromatin regions via ATAC-seq up to 15 kilobases (kb) away from gene bodies.

In an experimental example, using an expression atlas from maize, an embodiment of a transformer-based architecture was trained to predict the relative expression of each gene in six diverse tissues (i.e., leaf, embryo, anther, cob/inflorescence, endosperm, and root) given the set of DNA sequence starting 15 kilobases upstream from the annotated transcription start site and extending 15 kilobases downstream of the annotated transcription end site. The accuracy of predicted expression on holdout test genes belonging to families excluded from the training set exceeded the performance of three different controls. The best performing control was a test referred to herein as the Fortune Cookie Test, which measured how well the predictions made from the sequence of a randomly selected controlled gene predicted the observed expression pattern for a gene of interest. The Fortune Cookie Test significantly outperformed more conventional controls, likely as a result of common patterns of expression across the six tissues that were evaluated, which suggests that simpler models may over-estimate the true predictive performance of models trained to predict gene expression patterns from DNA sequence or other data types. A gradient-based approach was used to calculate the saliency of the input sequence in predicting relative tissue expression.

For this experimental example, the average saliency for 3,515 test genes not included in the trained dataset was calculated across upstream, gene body, and downstream regions. Saliency tended to increase substantially at the annotated transcription start site, with higher average saliency downstream of the annotated gene body than upstream, consistent with previous reports that three prime regions appear to play significant roles in determining, or at least predicting, patterns of gene expression. The spike in saliency at the annotated transcription start site appeared to be partially attributable to memorization rather than purely de novo identification of the transcription start site. When the model was used to predict gene expression patterns with sequences containing only 12, 9, 6, or 3 kilobases of upstream sequence, significant increases in saliency were still observed 15 kilobases downstream of the start of the sequence provided. However, distributions of predicted versus observed relative expression levels differed significantly from the most stringent control (Fortune Cookie Test) (p<0.001, Mann-Whitney U test). Beyond overall averages, saliency maps for individual genes exhibited diverse patterns of sharp peaks, indicating the predictions of the model depended on the DNA sequence in different regions surrounding the gene body for different individual genes. The overlap between saliency and conserved noncoding sequences were evaluated using a set of 230 Bigfoot genes associated with an usually large number of conserved noncoding sequences in both maize gene models and the rice orthologs of those same maize genes. Maize-rice conserved non-coding sequences often co-localized with spikes in saliency maps and the median base pair within a conserved non-coding sequence exhibited significantly higher saliency than the remainder of upstream or downstream regions (p<0.0001 and p<0.0001) respectively, t-test for independent samples). Similarly, overlap between saliency and open chromatin regions via ATAC-seq was evaluated. ATAC-seq peaks localized with spikes in saliency maps. The median base pair within ATAC-seq peaks exhibited significantly higher saliency than the remainder of upstream or downstream regions (p<0.0001 and p<0.0001 respectively, t-test for independent samples) calculated by averaging the saliency for 15,000 base pair upstream sequence, the binned saliency of each gene model ranging from the transcription start site to the transcription terminator site and, 15,000 base pairs downstream of each gene model. Higher saliency values were observed near the five prime proximal end of the binned gene bodies and proximal upstream regions of the transcription start site.

Based on these experimental results, it was determined that training a machine learning model to predict relative, rather than absolute, expression across multiple tissues based on the DNA sequence surrounding the gene and using saliency to assess which parts of the DNA sequence contributed the most to the model's predictions, enabled the accurate identification of known DNA regulatory regions. Applicant recognized that this approach enables the identification of previously unknown regulatory regions and provide guidance to efforts to engineer changes in gene expression—both absolute levels of gene expression and patterns of expression across cell types, tissues, environments, disease states—via targeted edits to the DNA-sequence at nucleotides identified via the saliency output of the trained machine learning model.

Applicant further recognized that, one of the key barriers to employing such a machine learning model has been that the memory requirements of employing attention-based machine learning algorithms for larger regions of sequence rapidly become intractable and unscalable. For example, in conventional approaches, calculating attention for a 15,000 base pair sequence requires considering all possible combinations of context (e.g., 225,000,000 in this case). As a result, efforts to train attention-based ML models for DNA sequence-based applications in plants have typically employed short regions, such as 1,000 base pairs, which is too small to capture proximal and distal promoter elements for many genes. To address these shortcomings, the techniques described herein employ a set of convolutional layers to create embeddings from the DNA sequence prior to providing the DNA sequence to the attention layers. Applicant recognized that this innovation enables the model to handle much larger amounts of DNA sequence with modest memory and computational resources. Applicant recognized that this improves the operation of a computing system upon which the model is deployed, enabling superior predictions while utilizing relatively fewer memory and processing resources relative to other gene expression predictions models.

Averaging across a given embedding of a DNA sequence might undesirably eliminate information on the spatial arrangement of regulatory DNA elements and damage model performance. However, counterintuitively and surprisingly, the transformer-based model described herein tolerated this averaging well, and the use of averaging across each embedding allowed the development of a transformer-based model capable of producing a constant set of outputs (in this case relative expression across a fixed set of tissues) from variable lengths of DNA sequence. Applicant recognized that predicting from variable lengths of DNA sequence is important for a number of reasons. For example, different applications may require understanding regulatory elements at different distances from the gene of interest, and it is infeasible to train separate models for each distance. Additionally, because the lengths of genes themselves are variable, if a fixed-length DNA sequence were required as input to the model, Applicant recognized that it would not be possible to include the full length of each gene without making other compromises to the model.

Present embodiments relate to systems and methods for constructing, training, and utilizing a large contextual gene sequence model to predict, from a DNA sequence of an organism, a pattern of mRNA and/or protein expression across different types of tissues of the organism, and to identify which regions of the DNA sequence are associated with regulation of the mRNA and/or protein expression across the different types of tissue of the organism. One such embodiment of a system is a machine learning model trained to predict a pattern of expression of a target gene contained in an input DNA sequence of an organism. The machine learning model includes a set of convolutional layers configured to generate embeddings of the input DNA sequence, a positional encoding layer configured to receive the embeddings of the input DNA sequence and to generate positional information for the embeddings of the input DNA sequence, a set of multi-headed attention layers configured to receive the embeddings of the DNA sequence and the positional information and to generate attention scores, and a set of fully connected output layers configured to receive the attention scores and provide an output indicating the relative abundance of expression of the target gene across a plurality of different tissue types of the organism.

In some embodiments, in forward operation, the machine learning model is configured to receive the input DNA sequence containing the target gene and to provide the output indicating the relative abundance of expression of the target gene across the plurality of different tissue types of the organism. In some embodiments, in reverse operation, the machine learning model is configured to receive the input DNA sequence containing the target gene and a second input indicating the relative abundance of expression of the target gene across the plurality of different tissue types of the organism, and is further configured to provide a second output including a respective saliency score for each of a plurality of nucleotides of the input DNA sequence located upstream and/or downstream of the target gene estimated via a gradient-based approach.

In some embodiments, the machine learning model includes a set of max pooling layers interposed between certain convolutional layers of the set of convolution layers, in which the set of max pooling layers and the embeddings generated by the set of convolutional layers are configured to reduce dimensions of the input DNA sequence, such that the machine learning model is capable of receiving a variable-length input DNA sequence. In some embodiments, each max pooling layer of the set of max pooling layers has a respective kernel size of 2 and a respective step size of 2. In some embodiments, the set of convolutional layers includes six convolutional layers, each convolutional layer having a respective kernel size of 15, and the six convolutional layers respectively having 1000, 500, 250, 500, 500, and 1000 feature maps. In some embodiments, each convolutional layer of the set of convolutional layers is followed by a Rectified Linear Unit (RELU) activation function.

In some embodiments, the set of multi-headed attention layers includes 5 multi-headed attention layers, in which each multi-headed attention layer includes 1000 embedding dimensions, 8 attention heads, and a skip connection incorporating the positional information from the positional encoding layer. In some embodiments, the set of fully connected output layers includes 3 fully connected output layers respectively having sizes of 4000, 1000, and 6. In some embodiments, the output contains a fixed number of values indicating the relative abundance of expression of the target gene across the plurality of different tissue types of the organism regardless of a length of the input DNA sequence. In some embodiments, the output indicates the relative abundance of expression of the target gene across the plurality of different tissue types of the organism in terms of relative abundance of messenger ribonucleic acid (mRNA) expression across the plurality of different tissue types of the organism, or relative abundance of protein expression of the target gene across the plurality of different tissue types of the organism.

One such embodiment of a method is a method of engineering expression of a target gene of a deoxyribonucleic acid (DNA) sequence of an organism. The method includes providing the DNA sequence containing the target gene as input to a trained machine learning model, and receiving, as output from the trained machine learning model, (i) relative predicted abundance of expression of the target gene across multiple types of tissues of the organism and (ii) a respective saliency score for each of a plurality of nucleotides of the DNA sequence located upstream and/or downstream of the target gene. The method includes selecting a portion of the plurality of nucleotides of the DNA sequence located upstream and/or downstream of the target gene having high saliency in predicting abundance of expression of the target gene. The method includes altering the portion of the plurality of nucleotides of the DNA sequence located upstream and/or downstream of the target gene, thereby altering the abundance of expression of the target gene in one or more of the multiple types of tissues of the organism.

In some embodiments, prior to providing the DNA sequence containing the target gene as input to a trained machine learning model, the method includes training a machine learning model to encode relationships between (i) DNA sequences of the organism or a related organism containing genes and (ii) the abundance of expression of the genes in the multiple types of tissues of the organism or the related organism, thereby to yield the trained machine learning model. In some embodiments, each of the DNA sequences of the organism or the related organism used for training respectively contain: a gene, at least 13 kilobases upstream of a transcription start site (TSS) of the gene, and at least 13 kilobases downstream of a transcription end site (TES) of the gene. In some embodiments, each of the DNA sequences of the organism or the related organism respectively contain: a gene, at least 15 kilobases upstream of a TSS of the gene, and at least 15 kilobases downstream of a TES of the gene. In some embodiments, the input DNA sequence contains the target gene, at least 13 kilobases upstream of a TSS of the target gene, and at least 13 kilobases downstream of a TES of the target gene. In some embodiments, the input DNA sequence contains the target gene, at least 15 kilobases upstream of the TSS of the target gene, and at least 15 kilobases downstream of the TES of the target gene.

In some embodiments, the trained machine learning model includes a set of convolutional layers and a set of attention layers, in which the set of convolutional layers is configured to create embeddings from the DNA sequence that are provided to the set of attention layers, such that the trained machine learning model is capable of receiving a variable-length DNA sequence as input. In some embodiments, the relative predicted abundance of expression of the target gene across the multiple types of tissues includes relative predicted abundance of messenger ribonucleic acid (mRNA) expression of the target gene across the multiple types of tissues of the organism, or relative predicted abundance of protein expression of the target gene across the multiple types of tissues of the organism.

In some embodiments, prior to altering the portion of the plurality of nucleotides of the DNA sequence located upstream and/or downstream of the target gene, the method includes evaluating a proposed alteration by providing a second DNA sequence containing the target gene and the proposed alteration of the portion of the plurality of nucleotides of the DNA sequence located upstream and/or downstream of the target gene as a second input to the trained machine learning model, and receiving, as a second output from the trained machine learning model, a tissues of the organism. In some embodiments, altering the portion of the plurality of nucleotides of the DNA sequence includes removing at least one nucleotide, modifying at least one nucleotide, replacing at least nucleotide with one or more naturally-occurring or artificial nucleotides, inserting at least one naturally-occurring or artificial nucleotide, or any combination thereof. In some embodiments, altering the portion of the plurality of nucleotides includes altering the portion of the plurality of nucleotides using CRISPR/Cas9. In some embodiments, the multiple types of tissues of the organism include leaf tissue, embryonic tissue, anther tissue, inflorescence tissue, endosperm tissue, root tissue, or any combination thereof.

One such embodiment of a method is a method of engineering the mRNA abundance of a target gene. The method includes training a machine learning model to predict the mRNA abundance of genes in one or more tissues, cell types, growing conditions, or other variation on experimental conditions given DNA sequence as input. The method includes employing the trained machine learning model to predict the mRNA abundance of a gene of interest given a set of DNA sequence associated with the gene. The method includes determining the saliency of individual DNA nucleotides within the DNA sequence used for prediction of the gene of interest. The method includes identifying regions within the DNA sequence provided for the gene of interest with high saliency in predicting the mRNA abundance of the gene of interest. The method includes altering the sequence of high saliency regions whether via gene editing or other suitable technologies.

In some embodiments, the model is trained specifically to predict the relative, rather than absolute, mRNA abundance of genes of interest across multiple tissues, cell types, growing conditions, or other variation on experimental conditions. In some embodiments, the model is employed to predict the effect of specific DNA sequence changes in high saliency regions and only DNA-sequence changes which are predicted to produce desired changes in mRNA abundance are introduced into the DNA sequence via gene editing or other technologies. In some embodiments, saliency is calculated by comparison to observed mRNA abundance levels for the gene of interest. In some embodiments, the saliency is calculated without reference to observed mRNA abundance. In some embodiments, the model is trained using mRNA abundance, and DNA sequence data from one species or a plurality of species and is employed to engineer changes in the mRNA abundance of a species included in the training dataset.

One such embodiment of a method is a method of using a machine learning model to predict the mRNA abundance of genes given DNA sequence as input. The method includes employing one or more convolutional layer to create embeddings of input DNA sequence. The method includes providing those embeddings as input to one or more attention layers. The method includes employing the output of the attention layer or layers to predict the mRNA abundance either directly or via additional layers.

In some embodiments, the output of the attention layer or layers is employed as an input to one or more fully connected layers prior to prediction of mRNA abundance. In some embodiments, a combination of convolutional layers and pooling are employed to reduce the dimensions of the input DNA sequence. In some embodiments, averaging within embeddings produced by the attention layer or layers is used to produce a fixed number of outputs from the output layer, regardless of the length of the input DNA sequence provided. In some embodiments, the model is trained to predict features or characteristics of genes other than mRNA abundance.

Aspects and advantages of these exemplary embodiments and other embodiments are discussed in detail herein. Moreover, it is to be understood that both the foregoing information and the following detailed description provide merely illustrative examples of various aspects and embodiments, and are intended to provide an overview or framework for understanding the nature and character of the claimed aspects and embodiments. Accordingly, these and other objects, along with advantages and features of the present disclosure, will become apparent through reference to the following description and the accompanying drawings. Furthermore, it is to be understood that the features of the various embodiments described herein are not mutually exclusive and may exist in various combinations and permutations.

### BRIEF DESCRIPTION OF THE DRAWINGS

The accompanying drawings, which are included to provide a further understanding of the embodiments of the present disclosure, are incorporated in and constitute a part of this specification, illustrate embodiments of the present disclosure, and together with the detailed description, serve to explain principles of the embodiments discussed herein. No attempt is made to show structural details of this disclosure in more detail than may be necessary for a fundamental understanding of the embodiments discussed herein and the various ways in which they may be practiced. According to common practice, the various features of the drawings discussed below are not necessarily drawn to scale. Dimensions of various features and elements in the drawings may be expanded or reduced to more clearly illustrate embodiments of the disclosure.

FIG. 1A is a graphical representation of a comparison of differing lengths and regions of contextual gene input sequence from previously published sequence-based models relative to an example implementation of the large contextual gene sequence model, according to an embodiment.

FIG. 1B is a graphical representation indicating Spearman rank correlation coefficients for all pairwise correlations between maize tissue types for 35,262 gene models, according to an embodiment.

FIG. 1C is a graphical representation of density plots representing the distribution of Spearman's rank coefficients of the three controls and the actual distribution as previously described for all test genes, according to an embodiment.

FIG. 2A is a graphical representation demonstrating an example of the distribution of maize-rice conserved noncoding sequences and saliency scores for a maize gene encoding a Cyclin D6 protein in v2 of the maize reference genome, according to an embodiment.

FIG. 2B is a graphical representation of the distribution of median max-normalized saliency scores for nucleotides located within and outside conserved noncoding sequences in the upstream and downstream regions of maize Bigfoot genes with at least one annotated conserved noncoding sequence present in the respective regions, according to an embodiment.

FIG. 2C is a graphical representation of an example of the distribution of ATAC-seq peaks and saliency scores for a maize gene encoding a domain of unknown function protein in v4 of the maize reference genome, according to an embodiment.

FIG. 2D is a graphical representation of the distribution of median max-normalized saliency scores for nucleotides located within and outside of the ATAC-seq peaks in the upstream and downstream regions of maize Bigfoot genes with at least one ATAC-seq peak present in the respective region, according to an embodiment.

FIG. 2E is a graphical representation of a plot of local z score based on the results of a permutation test assessing the association between maize conserved noncoding sequences or maize chromosome accessibility/ATAC peaks and high saliency scores genomic intervals of maize, according to an embodiment.

FIG. 2F is a graphical representation of a plot of local z score based on the results of a permutation test assessing the association between DNA Affinity Purification Sequencing (DAP-seq) peaks of multiple maize transcription factors and high saliency scores maize genomic intervals using genome-wide randomization of high saliency scores intervals, according to an embodiment.

FIG. 3A is a graphical representation of an example of the distribution of sorghum-rice conserved noncoding sequences and saliency scores for a sorghum gene encoding an uncharacterized protein in v1.4 of the sorghum reference genome, according to an embodiment.

FIG. 3B is a graphical representation of the distribution of median max-normalized saliency scores for nucleotides located within and outside of the conserved noncoding sequences in the upstream and downstream regions of sorghum Bigfoot genes with at least one annotated conserved noncoding sequence present in the respective regions, according to an embodiment.

FIG. 3C is a graphical representation of an example of the distribution of ATAC-seq peaks and saliency scores for a sorghum gene encoding a lipase in v3 of the sorghum reference genome, according to an embodiment.

FIG. 3D is a graphical representation of the distribution of median max-normalized saliency scores for nucleotides located within and outside of the conserved noncoding sequences in the upstream and downstream regions of sorghum Bigfoot genes with at least one annotated conserved noncoding sequence present in the respective regions, according to an embodiment.

FIG. 3E is a graphical representation of a plot of local z score based on the results of a permutation test assessing the association between sorghum conserved noncoding sequences or sorghum chromosome accessibility/ATAC peaks and high saliency scores genomic intervals of sorghum using genome-wide randomization of high saliency scores intervals, according to an embodiment.

FIG. 3F is a graphical representation of a plot of local z score based on the results of a permutation test assessing the association between DAP-seq peaks of multiple sorghum transcription factors and high saliency scores sorghum genomic intervals using genome-wide randomization of high saliency scores intervals, according to an embodiment.

### DETAILED DESCRIPTION

The present disclosure describes various embodiments related to systems and methods for constructing, training, and utilizing a transformer-based machine learning model for genetic research and engineering applications. The description may use the phrases “in certain embodiments,” “in various embodiments,” “in an embodiment,” or “in embodiments,” which may each refer to one or more of the same or different embodiments. Furthermore, the terms “comprising,” “including,” “having,” and the like, as used with respect to embodiments of the present disclosure, are synonymous. The term “plurality” as used herein refers to two or more items or components. The terms “about” or “approximately” are defined as being close to as understood by one of ordinary skill in the art. In one non-limiting embodiment, these terms are defined to be within 10%, preferably within 5%, more preferably within 1%, and most preferably within 0.5%. The use of the words “a” or “an” when used in conjunction with any of the terms “comprising,” “including,” “containing,” or “having,” in the claims or the specification may mean “one,” but it is also consistent with the meaning of “one or more,” “at least one,” and “one or more than one.”

### Glossary

Deoxyribonucleic acid (DNA) sequence: Information on an existing or hypothetical order of deoxyribonucleic acid nucleotides. The four common deoxyribonucleic acid nucleotides are typically represented by the letters A (adenine), T (thymine), C (cytosine), and G (guanine), but DNA sequences can also contain Xs or Ns, indicative of masked sequences or gaps where the identity of nucleotides are not known, as well as other information representing either ambiguous positions (e.g. those where two or more different nucleotides may be present) or artificial deoxyribonucleic acid nucleotides beyond the four typically found in natural systems.

Gene: A gene is the smallest unit of inheritance and contains the instructions needed to build and maintain living organisms. It contains of sequence of nucleotides made up of nucleic acid. Genes are contained within genomes, which are much longer sequences of nucleotides. Genes are frequently represented by gene models, which are a set of numerical descriptions of the start and stop positions of the sequence of individual genes within the genome.

Messenger Ribonucleic acid (mRNA) abundance: mRNAs are polymers of ribonucleic acid nucleotides that represent an intermediate step between protein coding genes encoded in a deoxyribonucleic acid genome and the amino acid-based polypeptides that constitute proteins. Different numbers of mRNA molecules will be transcribed from the same genes in different cell types, or in response to different stimuli. mRNA abundance is a measurement or estimate of the number of copies of mRNA produced by a given gene in a given sample, and can be calculated either in absolute terms, relative to abundance of the mRNA of another gene, or as an estimated proportion of all the mRNA transcripts produced within a given sample.

Open chromatin regions (OCRs): Also known as accessible chromatin regions, OCRs are areas of the genome where DNA is less tightly packed and can be accessed by regulatory proteins, influencing gene expression and other cellular processes.

Exons: Segments of DNA that contain the coding sequence for proteins.

Noncoding sequences: Segments of DNA that do not contain the coding sequence for proteins.

Machine Learning Model: A mathematical model that learns a representation of an input after finding associated patterns that connects inputs with its associated label. The learned representations are then used to predict the label of unseen or previously seen input.

Saliency: A measure of the importance of each input feature in determining the output of a model. Saliency can be calculated using a variety of methods and can be calculated both based on the difference between model predictions and a known set of ground truth outputs or based on the difference in two sets of predictions with varying inputs.

Layer: A step in a neural network where input data is transformed into a different output.

Attention layer: A layer in a neural network that assigns an attention weight or score to each dimension of input data based on its relevance to every other input dimension. This enables the neural network to detect the importance of a feature of an input in the context of all other input features. The attention scores are then used to assign higher scores to input features that contribute more to the final output.

Convolutional layer: A layer that uses matrices of weights (e.g., filters, kernels) that slide across the input to the layer, performing operations over each part of the input that it slides over (i.e., each matrix convolves the input). The operations generally extract patterns and relationships within the data.

Fully Connected Layer: A layer within a neural network where each neuron is connected to every other neuron in the previous layer.

Embedding: A vector of numeric values that are a transformed representation of some initial set of values.

Pooling: A process in neural networks that summarizes input data by reducing the spatial dimension along a specified axis of the data.

One-hot encoding: A technique used to convert categorical data into a binary format where each category is represented by a separate column with a 1 indicating its presence and 0s for all other categories.

Spearman's rank correlation coefficient: A coefficient, often denoted as ρ (rho), that measures the strength and direction of a monotonic relationship between two variables.

Bigfoot genes: Genes that are identified based on experimental evidence, correspond to large complex promoters, are often related to stress response and cell specialization, and have expression that tends to be difficult to understand.

Machine learning-based approaches to predicting gene expression levels and patterns from associated DNA sequence are advancing rapidly. In many cases, only sequences from the proximal noncoding regions (e.g., one kilobase up and downstream of annotated exons) are employed for prediction, despite extensive evidence that more distal noncoding regions play key roles in determining gene expression pattern. In contrast, present embodiments involve the use of a large contextual gene sequence model that utilizes sequence data from larger genomic intervals surrounding annotated genes to predict relative expression across a set of transcriptionally diverse tissues. Experimental results demonstrate that per-nucleotide saliency scores extracted from an embodiment of this large contextual gene sequence model are significantly associated with two different markers for functional noncoding regulatory sequence: the presence of conserved noncoding sequences and open chromatin regions. These results suggest that transformer-based architectures may provide a workable approach to guide efforts both to shape the transcriptional regulation of genes via gene editing beyond proximal promoter regions and understand the large proportion of standing phenotypic variation in populations attributable to genetic variances in noncoding regulatory sequence.

The experimental results presented herein are particular to the training and use of a large contextual gene sequence model (also described herein as an empirically-derived custom transformer-based model, or transformer-based model) to predict mRNA and/or protein expression in various plant tissues, as well as to predict saliency of DNA sequence regions with respect to the regulation of such mRNA and/or protein expression, in maize and sorghum plants. However, it should be appreciated that the techniques described herein may be applied to any multicellular organisms having differentiated tissues, including any plant, animal, or fungal species. It may be further appreciated that the large contextual gene sequence models described herein can be used in various manners to facilitate better understanding of protein expression and regulation in various organisms and to predict how changes to the DNA sequence may affect protein expression across different types of tissue.

For example, in forward operation, an embodiment of the large contextual gene sequence model may be provided with a DNA sequence of an organism as input, and may provide an output predicting mRNA and/or protein expression across various types of tissue of the organism. It is noted that this use case is valuable to projects exploring targeted genetic modification intended to affect protein expression in particular types of tissue of the organism without causing deleterious disruption to other types of tissue. For example, the “forward” operation of the model may be used to rapidly predict the relative protein expression across different tissue types that may result from potential alterations of a regulatory region of the DNA sequence, which may enable a researcher to more quickly identify and vet potential DNA sequence modifications for experimental testing. This approach can save a substantial amount of experimental time, resources, and costs, enabling researcher to avoid experimental efforts that are less likely to yield the desired levels of protein expression in particular types of tissue. It should be noted that the modification to the DNA sequence may include any suitable modification, such as substituting one or more base pairs with other naturally-occurring or non-naturally occurring base pairs, inserting additional base pairs, removing base pairs, and/or modifying (e.g., methylating, demethylating) one or more base pairs of the DNA sequence.

In reverse operation, an embodiment of the large contextual gene sequence model may be provided with the relative expression of mRNA and/or proteins across various tissues of an organism and a DNA sequence, and may provide an output predicting saliency of particular portions of the DNA sequence of the organism in the regulation of such mRNA and/or protein expression using a gradient-based approach. It is noted that this use case is valuable to projects exploring where DNA sequence modifications should be made, as well as what the modifications should be, to affect protein expression in particular types of tissue of the organism. In some use cases, the relative expression of mRNA and/or proteins across the various tissues of the organism may be experimentally determined, and the saliency of various portions of the DNA sequence output by the model may be used to better understand relative protein expression across the various tissue types of the organism. In some use cases, the relative expression of mRNA and/or proteins across the various tissues of the organism may reflect desired levels of expression to achieve a particular change or improvement in the organism, and the saliency of various portions of the DNA sequence output by the model may be used to guide researcher to the portions of the DNA sequence to be modified to affect this change or improvement. In some use cases, the relative expression of mRNA and/or proteins across the various tissues of the organism provided as input to the “reverse” operation of the model may be the predicted relative expression of mRNA and/or proteins previously provided as output generated by the “forward” operation of the model. Additionally, in some implementations, as additional experimental data is obtained, for example, by making modification to the DNA sequence of the organism and experimentally assessing mRNA and/or protein expression in the various tissues of the organism, this experimental data then may be used to train and/or fine tune the model, further enhancing the prediction accuracy of the model.

### Experimental Example

For the experimental data presented herein, an embodiment of a large contextual gene sequence model (a transformer-based model) was trained to predict the relative expression of individual maize genes across six highly differentiated tissues using the sequence of the genomic interval starting 15 kilobases upstream of the gene's transcription start side (TSS) and extending 15 kilobases downstream of the gene's transcription end site (TES). Gene-family guided-splitting was employed to reduce data leakage between training and test data. To evaluate model performance, a Fortune Cookie Test as described herein was adopted, as it was observed that this control significantly outperformed more conventional controls, likely as a result of repeated patterns of tissue-specific expression across unrelated genes. The trained large contextual gene sequence model significantly outperformed the Fortune Cookie Test, as assessed via average Spearman's rank coefficient (ρ) between predicted and observed gene expression in 3,515 hold out test genes (7.349×10⁻⁴¹, Mann Whitney U test). The predictive performance of the trained large contextual gene sequence model declined rapidly when smaller regions of noncoding sequence surrounding the gene of interest were employed for prediction and only exceeded that of controls when 13 kilobases or more of surrounding noncoding sequence was provided to the model.

### Materials and Methods for Experimental Example

An embodiment of an empirically-derived custom transformer-based model was trained to predict the relative expression values of maize genes across six tissue types: leaf, root, anthers, immature cob, embryo, endosperm. For each gene, expression data was max normalized as a proportion of the maximum expression value across the six tissue types for that gene. Maize genes from the B73 RefGen V5 reference genome were clustered into 10,738 unique gene families and whole gene families were assigned to training, testing, and validation datasets, such that the total number of genes in each category approximated an 80%/10%/10% split. The input sequence for predicting each gene was the genomic interval beginning 15,000 base pairs upstream of the annotated TSS of the primary transcript and ended 15,000 base pairs downstream of the annotated TES. Per nucleotide saliency in maize was calculated by taking the gradient of the loss with respect to the input sequence where loss values were calculated via comparison of the predicted and observed relative expression profiles for the gene of interest. Expression and per nucleotide saliency values in sorghum were calculated similarly with the modification that loss was calculated relative to expression in six sorghum tissues identified as most similar to the six maize tissue-level expression datasets used in this study, based on the degree of correlation in the absolute level of expression of syntenic orthologs between maize and sorghum expression datasets. Data on the positions of conserved noncoding sequences between the rice genome and syntenic orthologs in maize and sorghum were obtained from previous publications (see G Turco et. al, Front. plant science 4, 52502 (2013)). As these annotated conserved non-coding sequences (CNS) were identified in earlier drafts of the maize and sorghum reference genomes, new saliency values were calculated using genomic intervals extracted from these earlier versions of the maize and sorghum reference genomes. Open chromatin windows defined via ATAC-seq were taken from leaf tissue in sorghum and leaf tissue in maize.

### Extracting and Processing Sequence for Gene Models

For the experimental example, the version 5 maize reference genome and annotation files were downloaded from phytozome. The DNA sequence for 39,756 gene models was extracted from the Zea mays reference genome. Sequences on the reverse strand were converted to the forward form of the gene. The sequence for 15,000 base pairs upstream of the TSS and 15,000 base pairs downstream of the TES was attached to each gene model. The concatenated sequence for each gene model was one-hot encoded before input into the transformer-based model.

### Extracting and Processing of Tissue-Specific Expression of Gene Models

For the experimental example, the expression of 39,756 maize gene models across 57 tissues was extracted from the MaizeGDB website. The tissue samples were extracted from a B73 inbred genotype. Sequenced reads were mapped to version 2 of the B73 reference genome assembly (see P S Schnable, et al., Science 326, 1112-1115 (2009)) using previously described methods (see R S Sekhon, et al., PloS one 8, e61005 (2013)). 33,197 sorghum gene models across 49 tissues were compiled from EMBL's European Bioinformatics Institute (EMBL-EBI) website, which was a compilation of data from seven previously published datasets. Pearson correlation coefficient values were derived for all maize tissue types and sorghum tissue types using the expression of 11,108 maize genes and the sorghum orthologs of the maize subgenome 1. Six highly correlated identical tissue types of maize and sorghum were selected. The name of the maize tissue types listed were ‘V7 Tip of transition leaf maize’, ‘Embryo 22 DAP’, ‘Anthers R1’, ‘V18 Immature cob’, ‘Endosperm 24 DAP’, ‘Primary Root 6 days after sowing GH’ for maize and that of sorghum was ‘flag leaf’, ‘plant embryo’, ‘anther’, ‘inflorescence size 1 to 10 millimeter, inflorescence’, ‘20 days after pollination, endosperm’ and ‘seedling developmental stage, root’. Tissue types are referred to herein as the leaf, embryo, anther, cob/inflorescence (IM), endosperm, and root. For this experimental example, tissue expression was measured in units of fragments per kilobase of transcript per million (FPKM).

### Architecture and Training of Transformer-Based Model

For the experimental example, an embodiment of an empirically-derived custom transformer-based model with input convolutional layers was implemented in python using Pytorch (V2.1.0) software. For this embodiment, there were six convolutional layers, all having kernel sizes of 15, and respectively having 1000, 500, 250, 500, 500, and 1000 feature maps. The 2nd, 4th, and 6th convolutional layers were each followed by a Rectified Linear Unit (RELU) activation function, a max pooling layer of a kernel size of 2 and step size of 2, then a dropout layer of 0.25 before the succeeding convolutional layers. All other convolutional layers were followed only by a RELU activation function. The output of the final convolutional layer was fed into a positional encoding layer, and then to five multi-attention layers, each having 1000 embedding dimensions, 8 attention heads, and a skip connection incorporating information from the positional encoding layer. Layer normalization was performed on each multi-head attention layer. Finally, the output of the series of multi-attention heads were fed into three fully connected layers of sizes 4000, 1000, and 6, respectively. A Binary Cross Entropy Loss Function, which took logits as input and applied a sigmoid activation function, was used to calculate the loss between the predicted and observed expression profile of each gene. An Adam optimizer was used to optimize the weights of the model towards a lower loss, using a starting learning rate of 0.000001. The input expression for each tissue type in the expression profile for each gene model was max normalized, as a proportion of the max expression across all tissues for each gene. It may be appreciated that, while the preceding described the embodiment of the transformer-based model used in this experimental example, in other embodiments, the structure, parameters, and/or optimization of the transformer-based model may vary.

### Family Guided Gene Splitting for Datasets

For the experimental example, the full maize dataset was split into training, validation, and testing data sets. These splits were guided according to gene families. Maize gene families were extracted from PLAZA (see M Van Bel, et al., Nucleic Acids Res. 50, D1468-D1474 (2022)) where there were 11,410 unique gene families ranging from families of 352 to single-family genes for 39,756 genes. The gene family dataset was subset to have 35,262 genes with 10,738 unique gene families ranging from 239 genes per family to families with just single genes. Each dataset contained genes in which families were only present in that specific data (i.e., no gene family was shared among the three datasets). The training, validation, and test sets contained 28,208, 3,527, and 3,527 genes for 4,403, 2,808, and 3,527 unique families, respectively. Furthermore, input sequences with more than 90,000 base pairs were skipped leaving 28,076, 3,521 and 3,515 genes for training, validation, and test sets, respectively.

### Evaluation of Model

For the experimental example, three random/baseline distributions were derived to compare against the distributions of the Pearson correlation coefficient values between observed and predicted expression profiles on the test dataset. Each of these distributions were the Pearson correlation coefficient values of random expression profiles and observed expression profiles for each gene. Distribution one took the predictions of the model on test data and for each gene, the expression of the four cell types were randomly shuffled. Distribution two took the whole observed expression profiles of each gene and compared those profiles with random observed profiles of other genes. Distribution three took the whole predicted expression profile of each gene and randomly assigned them to other genes. To evaluate the performance of the model on different parts of the input sequence, regions of the input sequence were masked, and the regions of interest were left unmasked. Masking refers to assigning all 0 values for the one-hot encoded value for each nucleotide of the masked region of the sequence. For the upstream region, any sequence after the transcription start site of the input sequence was masked before input into the model. For the gene body, 15,000 base pairs upstream of the transcription start site and 15,000 base pairs downstream of the transcription end site was masked. For the downstream region, all input sequence upstream of the transcription end site was masked. Finally, sequence that was neither 15,000 base pairs upstream of the transcription start site, 1000 base pairs downstream of the transcription start site, 1000 base pairs upstream of the transcription end site and 15,000 base pairs downstream of the transcription start site was masked to get a representation of the majority of proximal regulatory regions of the gene.

### Data Sources for Open Chromatin, Conserved Non-Coding Sequence and Highly Conserved Genes

For the experimental example, conserved non-coding sequences (CNS) for maize and sorghum were derived from previously published data (see G Turco, et al. Science 4, 52502 (2013)). Maize CNS were determined using a pipeline comparing sequence from version 2 of the maize reference genome and version 6 of the rice reference genome. 286 gene models in maize termed as maize Bigfoot genes (pairs of genes that had at least four conserved non-coding sequence over a non-coding five prime, plus three prime region of at least 4 kb and having at least one conserved non-coding sequence for every 1 kb of non-coding region) was subset for gene models whose tissue expression across the six tissue types was available for version 5 of the same gene model, totaling 230 Bigfoot genes. Gene annotation files for version 2 of the maize genome were used to extract gene model sequence with a padding of 15,000 base pairs (bp) upstream and 15,000 bp downstream of the annotated transcription start site and transcription end site, respectively. CNS for sorghum version 1 of the reference genome and version 6 of the rice genome were used. 332 sorghum Bigfoot genes were subset for genes having expression values when mapped to their version 3, leaving 242 total genes. Annotation files and sorghum version 1 reference genome was downloaded from CoGE comparative genomics toolbox (see E Lyons et al., Trop. Plant Biol. 1, 181-190 (2008)) and sequence for each gene model along with gene padding extracted as described above. ATAC-seq for version 4 of the maize reference genome was derived from the publicly accessible plant epigenome browser (see Z Lu, et al., Nat. Plants 5, 1250-1259 (2019)). Sequence and annotation files was downloaded from phytozome (see D M Goodstein, et al., Nucleic acids research 40, D1178-D1186 (2012)). ATAC-seq for version 3 of the sorghum reference genome was derived from previously published work (see C Zhou, et al., Plant Commun. 2 (2021)). Sequence and annotation files were downloaded from phytozome.

### Saliency Maps

For the experimental example, sequences for genes of interest were parsed from reference genomes with 15,000 bp padding upstream of the TSS and 15,000 bp downstream of the TES. Each sequence was pre-processed for input as described in model training. The relative expression of values of the input sequence was then predicted. The loss values between the predicted and observed relative expression values were determined by the model loss function. The gradient of the loss with respect to the input sequence was derived for each input nucleotide sequence and was referenced as the saliency score. Ambiguous nucleotides (X and N) were masked for downstream analyses. Normalized (relative) saliency scores for individual genes were determined by max normalization of all scores of the gene of interest. The median of normalized saliency scores within partitioned regions was determined for all Bigfoot genes. Saliency was partitioned into scores that overlapped conserved non-coding sequence in the upstream regions of the TSS, non-overlapping scores in the upstream regions of TSS, overlapping scores in the downstream region of the TES, non-overlapping scores in the downstream regions of the TES and scores in the untranslated regions, coding sequence, and intronic regions of the gene body. Median normalized saliency scores also partitioned into similar categories, substituting conserved noncoding regions with open chromatin regions as measured by ATAC-seq.

### Experimental Results

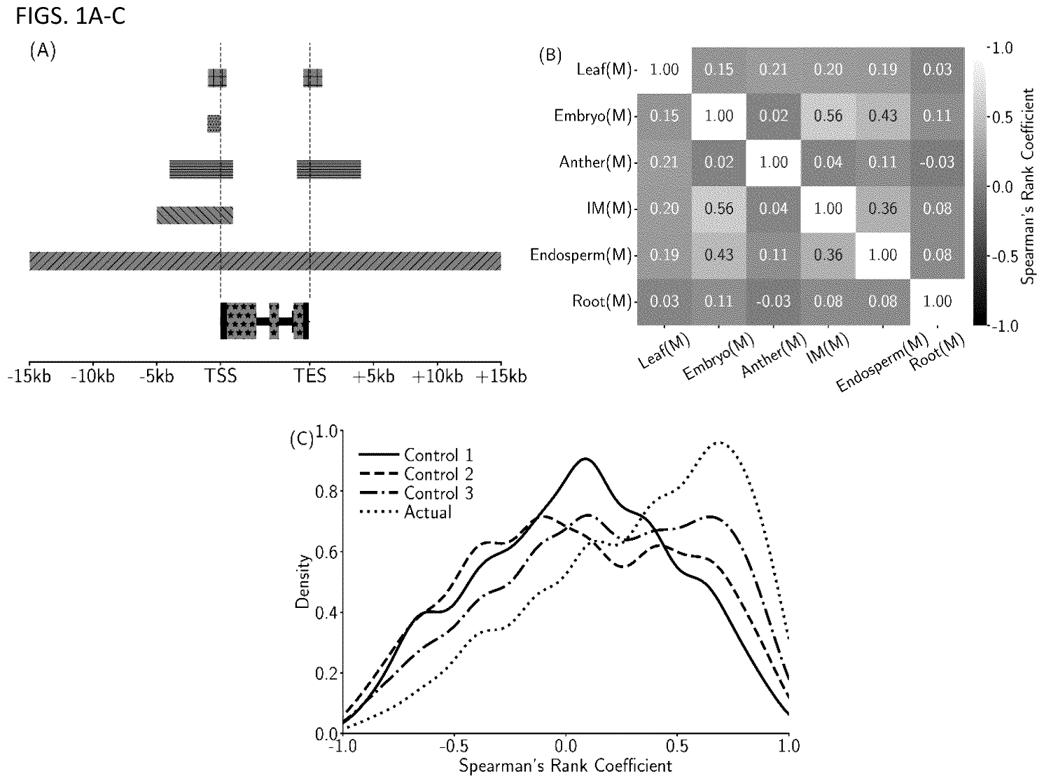

FIGS. 1A-1C present results of the experimental example demonstrating how an embodiment of a large contextual gene sequence model of the present approach outperforms baseline distributions in predicting relative expression values across plant organs. FIG. 1A is a graphical representation of a comparison of differing lengths and regions of contextual gene input sequence from previously published sequence-based models relative to the embodiment of the large contextual gene sequence model. The illustrated axis indicates the transcription start site (TSS) and the transcription end site (TES), and extends from 15 kilobases (kb) prior to the TSS to 15 kb after the TES. The top bar represents regions of contextual gene input sequence for a first sequence-based model previously published by Washburn et al. (2019), the second bar represents regions of contextual gene input sequence for a second sequence-based model previously published by Wrightsman et al. (2024), the third bar represents regions of contextual gene input sequence for a second sequence-based model previously published by Li et al. (2024), the fourth bar represents regions of contextual gene input sequence for a second sequence-based model previously published by Mendoza-Revilla et al. (2024), and the fifth (bottom) bar represents regions of contextual gene input sequence for the embodiment of the large contextual gene sequence model used in the experimental example.

FIG. 1B is a graphical representation indicating Spearman rank correlation coefficients for all pairwise correlations between maize tissue types for 35,262 gene models. The grey-scale bar represents the intensity according to Spearman's rank coefficient values for all heatmaps, in which darker colors represent negative correlation values, while lighter colors represent positive correlation values. FIG. 1C is a graphical representation of density plots representing the distribution of Spearman's rank coefficients of the three controls and the actual distribution as previously described for all test genes. Control 1 demonstrates Spearman's rank coefficients derived using the observed relative expression profile of each gene compared with a shuffled prediction profile of that same gene. Control 2 compares the observed relative expression profile of a gene with the observed expression profile of another gene. Control 3 compares the observed expression profile of a gene with the predicted expression profile of another random gene (Fortune Cookie Test). The plot labeled “Actual” demonstrates real Spearman rank coefficients, where the observed relative expression profile of a gene is compared with the predicted relative expression profile of the same gene.

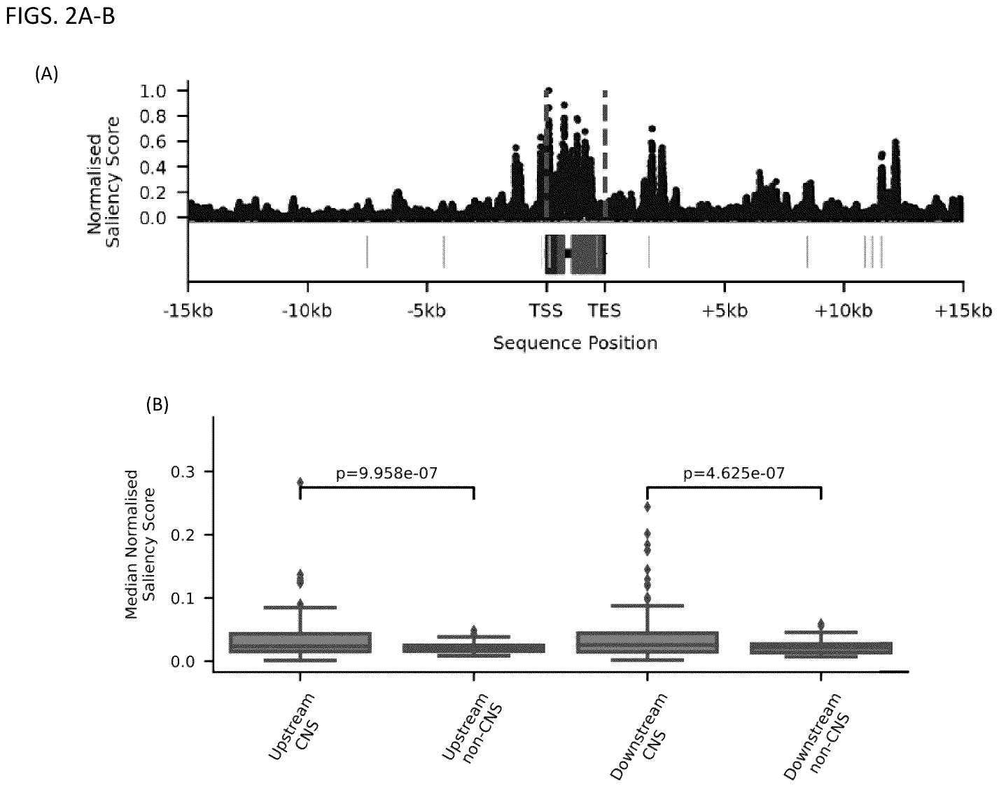

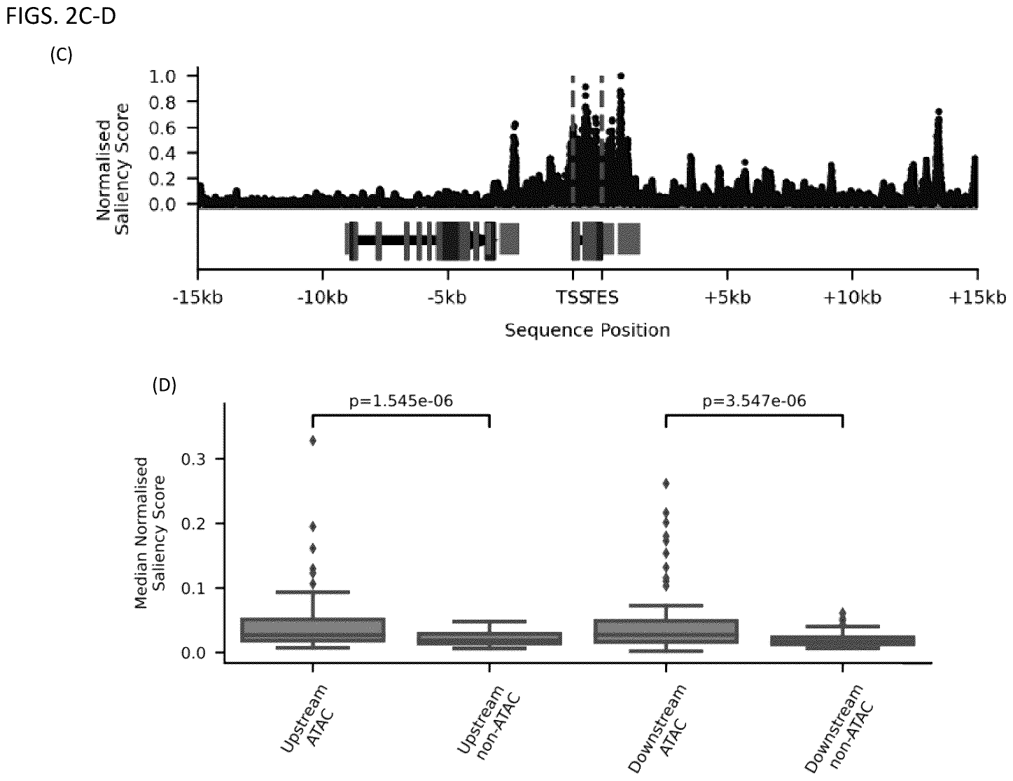

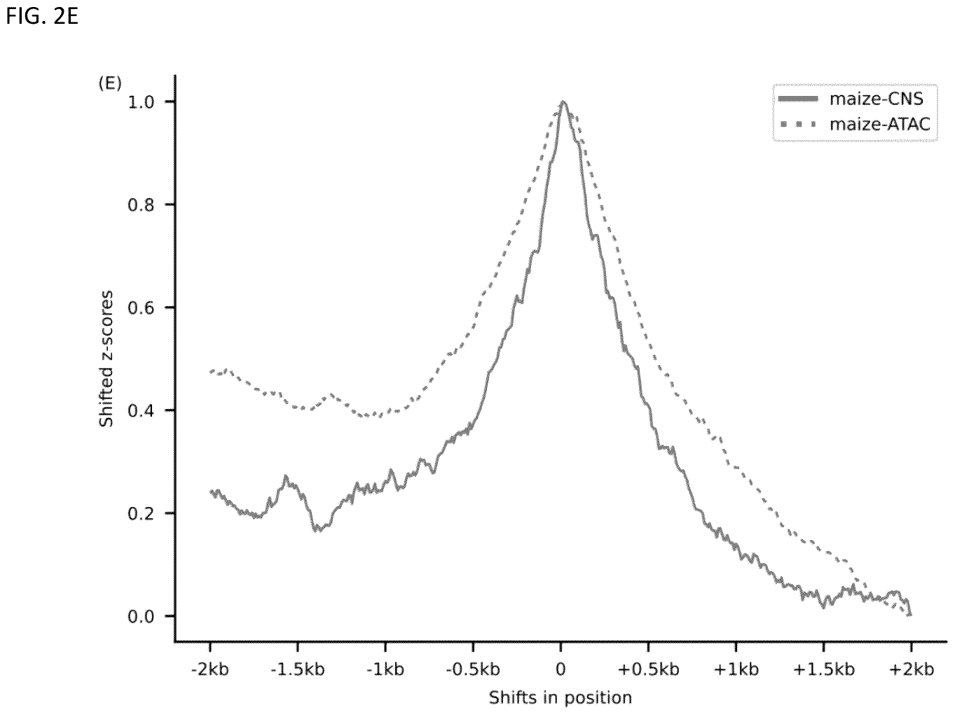

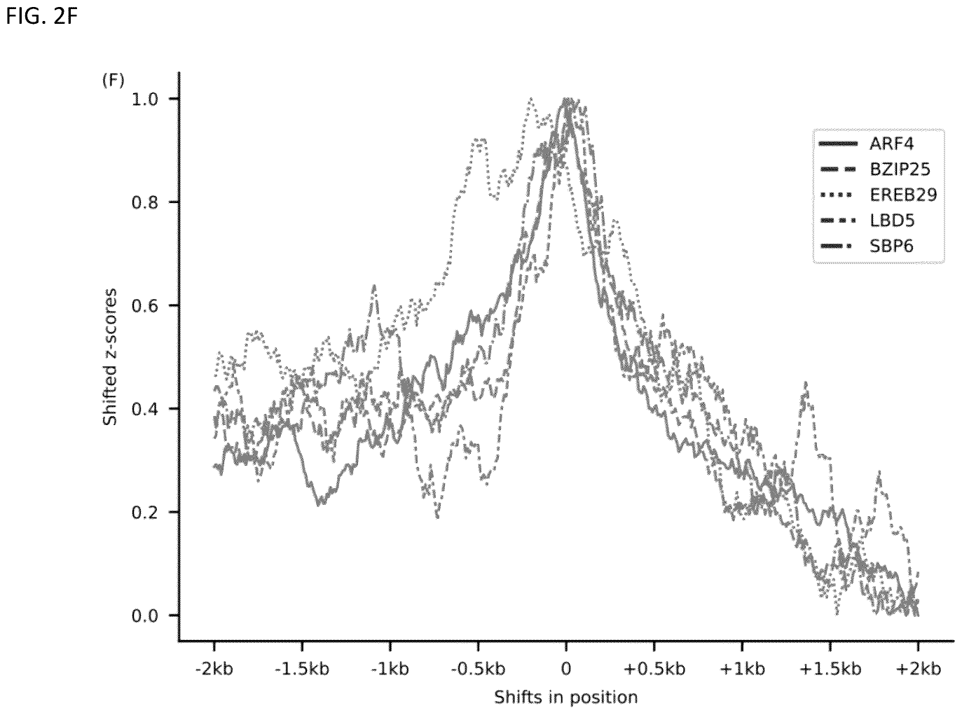

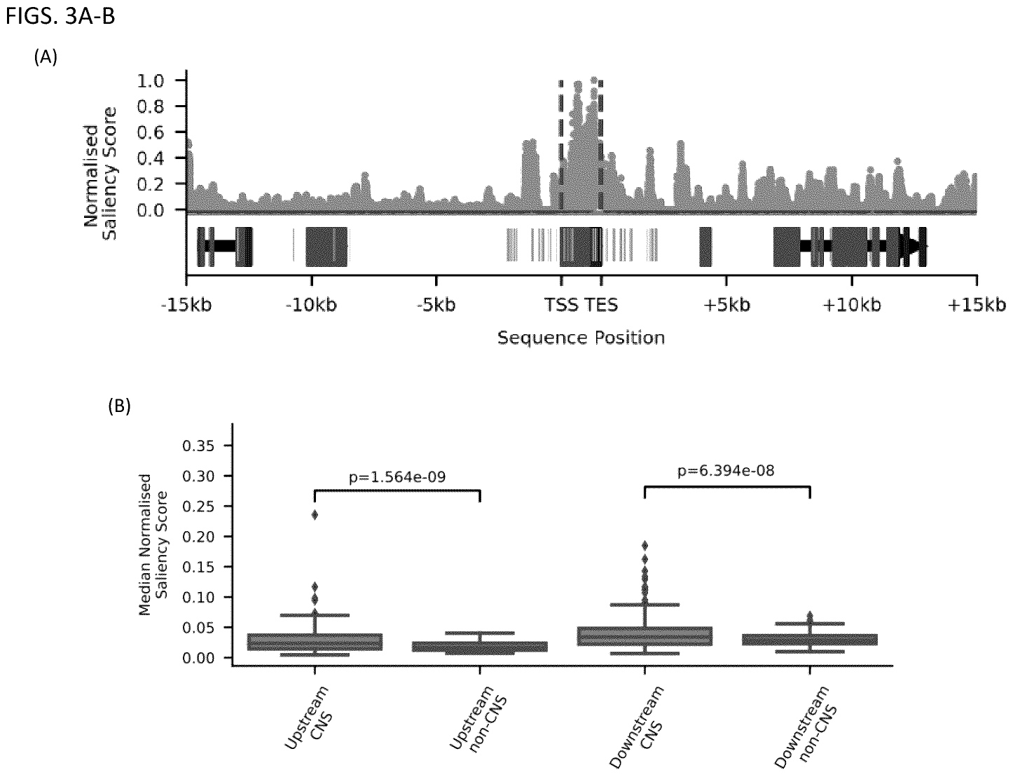

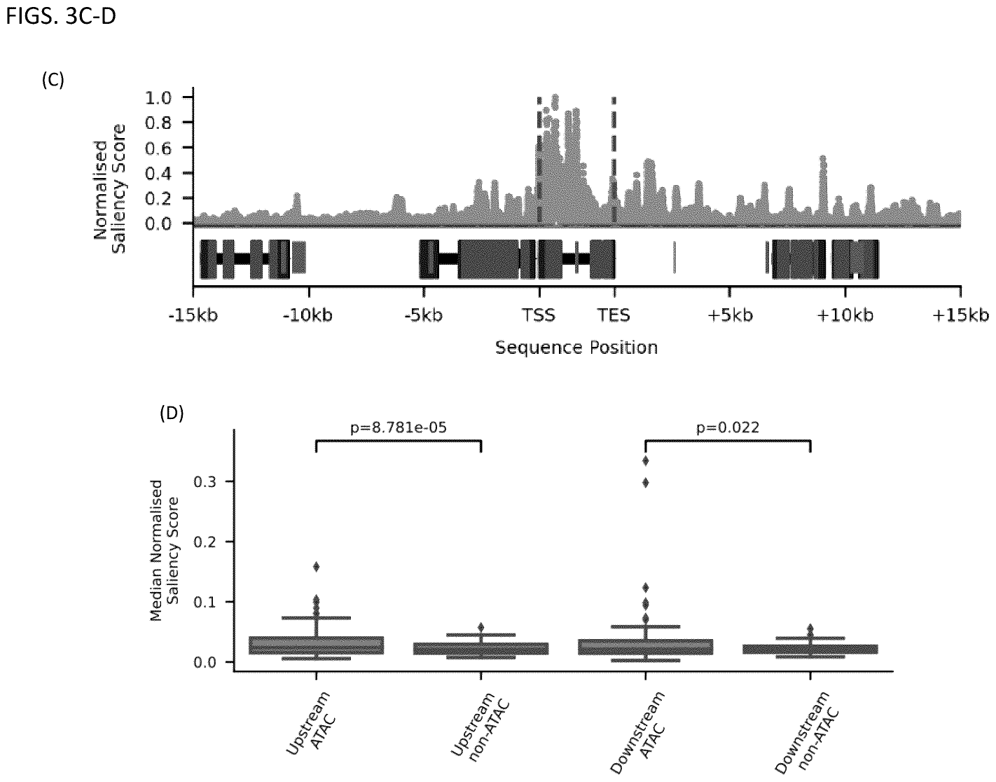

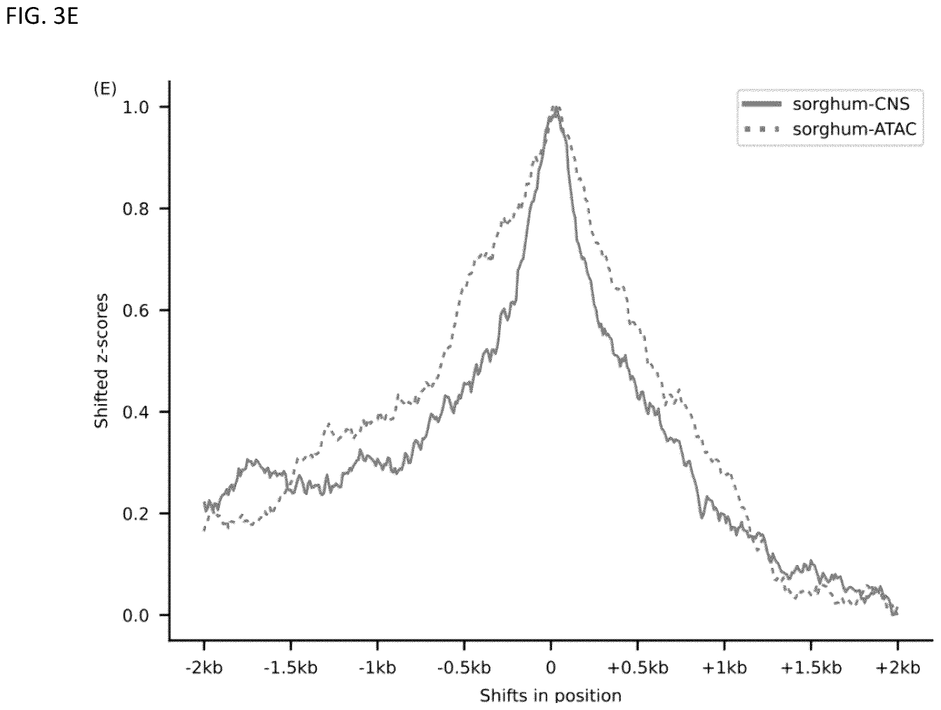

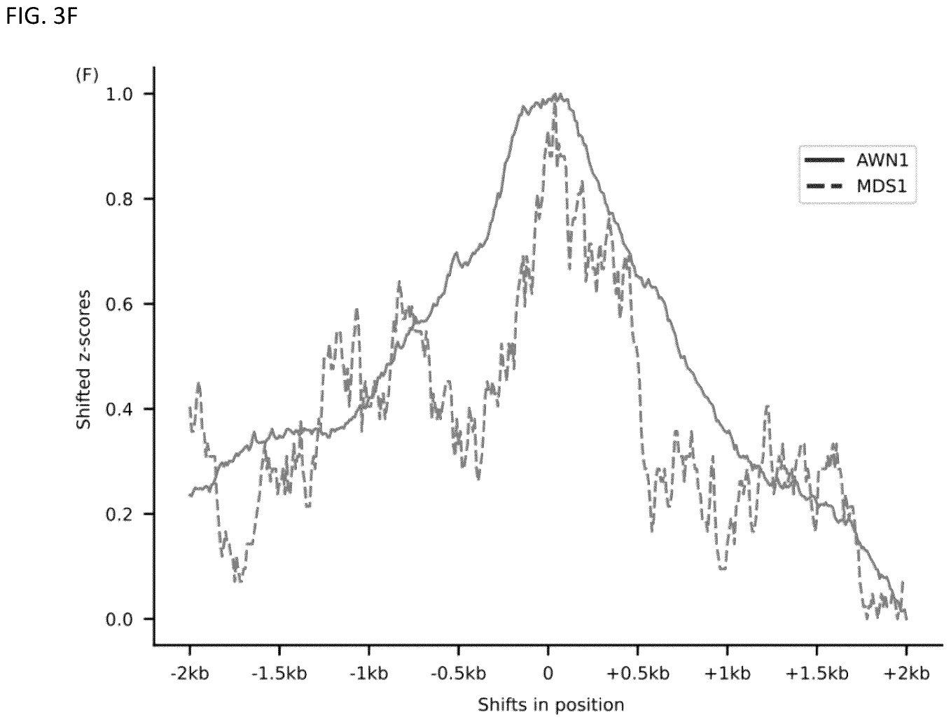

The overlap between the saliency of individual DNA nucleotides was evaluated when calculating relative tissue-level expression—estimated via a gradient-based approach—and two markers for DNA regulatory function, conserved noncoding sequences identified in comparisons between maize and rice and open chromatin regions identified via ATAC-seq taken from leaf tissue. Comparisons were conducted using a set of 230 Bigfoot genes that were associated with greater numbers of conserved noncoding sequences and typically exhibit more complex patterns of regulation and expression. A number of maize-rice conserved noncoding sequences co-localized with spikes in saliency (FIG. 2A), although not all spikes in saliency were tagged by conserved noncoding sequences, nor did all conserved noncoding sequences correspond to spikes in the saliency map. The median base pair within an upstream or downstream conserved non-coding sequence exhibited significantly higher saliency than median base upstream or downstream nucleotide, excluding conserved noncoding sequences, of the same gene (p=9.958×10⁻⁷, n=160 and p=4.625×10⁻⁷, n=163, respectively, paired t-test) (FIG. 2B). Similarly, many but not all ATAC-seq peaks co-localized with spikes in the saliency maps for individual genes (FIG. 2C) and the median base pair within ATAC-seq peaks exhibited significantly higher saliency than the median base pair in the remainder of the upstream or downstream regions of the same genes (p=1.545×10⁻⁶, n=106 and p=3.547×10⁻⁵, n=103, respectively, paired t-test) (FIG. 2D). The embodiment of the model employed for the experimental data presented herein was trained on data from only a single species, maize. Nevertheless, saliency scores calculated using the maize-trained model continued to be significantly associated with conserved noncoding sequence and open chromatin regions when the model was instead employed to predict relative patterns of tissue-level expression in a second related species, sorghum (FIGS. 3A-3D). A general trend towards higher saliency scores downstream rather than upstream of the target gene is consistent with previous models.

FIGS. 2A-2B present results from the experimental example demonstrating the association between saliency and marks of regulatory functions in maize. FIG. 2A is a graphical representation demonstrating an example of the distribution of maize-rice conserved noncoding sequences and saliency scores for a maize gene, GRMZM2G050933, encoding a Cyclin D6 protein in v2 of the maize reference genome. In the lower portion of FIG. 2A, the black arrow (partially occluded) indicates the positions and strands of annotated genes within the genomic interval used for prediction, the blue boxes indicate untranslated exonic sequences, the green boxes indicate translated exonic sequences, and the orange lines indicate the positions conserved noncoding sequences between maize and rice. In the upper portion of FIG. 2A, the blue dots indicate the max-normalized saliency score of the nucleotide at the corresponding position on the x-axis, and the vertical dashed red lines indicate the annotated TSS and TES of the gene of interest. FIG. 2B is a graphical representation of the distribution of median max-normalized saliency scores for nucleotides located within and outside conserved noncoding sequences in the upstream (−15 kb to TSS) and downstream (TES to +15 kb) regions of maize Bigfoot genes with at least one annotated conserved noncoding sequence present in the respective regions. P-values shown were calculated via paired, two-tailed t-tests (upstream region n=160, downstream region n=163).

FIG. 2C is a graphical representation of an example of the distribution of ATAC-seq peaks and saliency scores for a maize gene, Zm00001d006739, encoding a domain of unknown function (DUF260) protein in v4 of the maize reference genome. In the lower portion of FIG. 2C, the red boxes indicate the position of annotated ATAC-seq peaks, while all other features are as defined in FIG. 2A. FIG. 2D is a graphical representation of the distribution of median max-normalized saliency scores for nucleotides located within and outside of the ATAC-seq peaks in the upstream (−15 kb to TSS) and downstream (TES to +15 kb) regions of maize Bigfoot genes with at least one ATAC-seq peak present in the respective region. P-values shown were calculated via paired, two-tailed t-tests (upstream region n=106, downstream region n=103). In FIGS. 2B and 2D, shaded boxes represent the range between the 25th and 75th percentiles of median max-normalized saliency scores. Black solid lines inside shaded boxes indicate the median (50th percentile) value. Whiskers extend to either the minimum and maximum values or 1.5 times the interquartile range. In the latter case, any data points beyond the edge of the whiskers are indicated with filled circles.

FIG. 2E is a graphical representation of a plot of local z score based on the results of a permutation test assessing the association between maize conserved noncoding sequences (n=39,489) or maize chromosome accessibility/ATAC peaks (n=19,626) and high saliency scores genomic intervals of maize (n=183,870). FIG. 2F is a graphical representation of a plot of local z score based on the results of a permutation test assessing the association between DAP-seq peaks of multiple maize transcription factors (ARF4, n=23,173; BZIP25, n=64,716; EREB29, n=14,229; LBD5, n=10,130; SBP6, n=14,272) and high saliency scores maize genomic intervals using genome-wide randomization of high saliency scores intervals.

FIGS. 3A-3F present results of the experimental example demonstrating the association between saliency and marks of regulatory functions in sorghum. FIG. 3A is a graphical representation of an example of the distribution of sorghum-rice conserved noncoding sequences and saliency scores for a sorghum gene, Sb03g028390, encoding an uncharacterized protein in v1.4 of the sorghum reference genome. In the lower portion of FIG. 3A, the black arrows (partially occluded) indicate the positions and strands of annotated genes within the genomic interval used for prediction, the blue boxes indicate untranslated exonic sequences, the green boxes indicate translated exonic sequences, and the orange lines indicate the positions conserved noncoding sequences between maize and rice. In the upper portion of FIG. 3A, the blue dots indicate the max-normalized saliency score of the nucleotide at the corresponding position on the x-axis, and the vertical dashed black lines indicate the annotated TSS and TES of the gene of interest. FIG. 3B is a graphical representation of the distribution of median max-normalized saliency scores for nucleotides located within and outside of the conserved noncoding sequences in the upstream (−15 kb to TSS) and downstream (TES to +15 kb) regions of sorghum Bigfoot genes with at least one annotated conserved noncoding sequence present in the respective regions. P-values shown were calculated via paired, two-tailed t-tests (upstream region n=185, downstream region n=182).

FIG. 3C is a graphical representation of an example of the distribution of ATAC-seq peaks and saliency scores for a sorghum gene, Sobic.001G425300, encoding a lipase in v3 of the sorghum reference genome. In the lower portion of FIG. 3C, the red boxes indicate the position of annotated ATAC-seq peaks, while all other features are as defined in FIG. 3A. FIG. 3D is a graphical representation of the distribution of median max-normalized saliency scores for nucleotides located within and outside of the conserved noncoding sequences in the upstream (−15 kb to TSS) and downstream (TES to +15 kb) regions of sorghum Bigfoot genes with at least one annotated conserved noncoding sequence present in the respective regions. P-values shown were calculated via paired, two-tailed t-tests (upstream region n=185, downstream region n=182). In FIGS. 3B and 3D, shaded boxes represent the range between the 25th and 75th percentiles of median max-normalized saliency scores, while the black solid lines inside shaded boxes indicate the median (50th percentile) value. Whiskers extend to either the minimum and maximum values or 1.5 times the interquartile range. In the latter case, any data points beyond the edge of the whiskers are indicated with filled circles.

FIG. 3E is a graphical representation of a plot of local z score based on the results of a permutation test assessing the association between sorghum conserved noncoding sequences (n=34,463) or sorghum chromosome accessibility/ATAC peaks (n=11,637) and high saliency scores genomic intervals of sorghum (n=39,768) using genome-wide randomization of high saliency scores intervals. FIG. 3F is a graphical representation of a plot of local z score based on the results of a permutation test assessing the association between DNA Affinity Purification Sequencing (DAP-seq) peaks of multiple sorghum transcription factors (AWN1, n=14,814; MSD2; n=2,367) and high saliency scores sorghum genomic intervals using genome-wide randomization of high saliency scores intervals.

Present embodiments relate to systems and methods for constructing, training, and utilizing a large contextual gene sequence model to predict, from a DNA sequence of an organism, a pattern of mRNA and/or protein expression across different types of tissues of the organism, and to identify which regions of the DNA sequence are associated with regulation of the mRNA and/or protein expression across the different types of tissue of the organism. One such embodiment of a system is a machine learning model trained to predict a pattern of expression of a target gene contained in an input DNA sequence of an organism. The machine learning model includes a set of convolutional layers configured to generate embeddings of the input DNA sequence, a positional encoding layer configured to receive the embeddings of the input DNA sequence and to generate positional information for the embeddings of the input DNA sequence, a set of multi-headed attention layers configured to receive the embeddings of the DNA sequence and the positional information and to generate attention scores, and a set of fully connected output layers configured to receive the attention scores and provide an output indicating the relative abundance of expression of the target gene across a plurality of different tissue types of the organism.

In some embodiments, in forward operation, the machine learning model is configured to receive the input DNA sequence containing the target gene and to provide the output indicating the relative abundance of expression of the target gene across the plurality of different tissue types of the organism. In some embodiments, in reverse operation, the machine learning model is configured to receive the input DNA sequence containing the target gene and a second input indicating the relative abundance of expression of the target gene across the plurality of different tissue types of the organism, and is further configured to provide a second output including a respective saliency score for each of a plurality of nucleotides of the input DNA sequence located upstream and/or downstream of the target gene estimated via a gradient-based approach.

In some embodiments, the machine learning model includes a set of max pooling layers interposed between certain convolutional layers of the set of convolution layers, in which the set of max pooling layers and the embeddings generated by the set of convolutional layers are configured to reduce dimensions of the input DNA sequence, such that the machine learning model is capable of receiving a variable-length input DNA sequence. In some embodiments, each max pooling layer of the set of max pooling layers has a respective kernel size of 2 and a respective step size of 2. In some embodiments, the set of convolutional layers includes six convolutional layers, each convolutional layer having a respective kernel size of 15, and the six convolutional layers respectively having 1000, 500, 250, 500, 500, and 1000 feature maps. In some embodiments, each convolutional layer of the set of convolutional layers is followed by a Rectified Linear Unit (RELU) activation function.

In some embodiments, the set of multi-headed attention layers includes 5 multi-headed attention layers, in which each multi-headed attention layer includes 1000 embedding dimensions, 8 attention heads, and a skip connection incorporating the positional information from the positional encoding layer. In some embodiments, the set of fully connected output layers includes 3 fully connected output layers respectively having sizes of 4000, 1000, and 6. In some embodiments, the output contains a fixed number of values indicating the relative abundance of expression of the target gene across the plurality of different tissue types of the organism regardless of a length of the input DNA sequence. In some embodiments, the output indicates the relative abundance of expression of the target gene across the plurality of different tissue types of the organism in terms of relative abundance of messenger ribonucleic acid (mRNA) expression across the plurality of different tissue types of the organism, or relative abundance of protein expression of the target gene across the plurality of different tissue types of the organism.

One such embodiment of a method is a method of engineering expression of a target gene of a deoxyribonucleic acid (DNA) sequence of an organism. The method includes providing the DNA sequence containing the target gene as input to a trained machine learning model, and receiving, as output from the trained machine learning model, (i) relative predicted abundance of expression of the target gene across multiple types of tissues of the organism and (ii) a respective saliency score for each of a plurality of nucleotides of the DNA sequence located upstream and/or downstream of the target gene. The method includes selecting a portion of the plurality of nucleotides of the DNA sequence located upstream and/or downstream of the target gene having high saliency in predicting abundance of expression of the target gene. The method includes altering the portion of the plurality of nucleotides of the DNA sequence located upstream and/or downstream of the target gene, thereby altering the abundance of expression of the target gene in one or more of the multiple types of tissues of the organism.

In some embodiments, prior to providing the DNA sequence containing the target gene as input to a trained machine learning model, the method includes training a machine learning model to encode relationships between (i) DNA sequences of the organism or a related organism containing genes and (ii) the abundance of expression of the genes in the multiple types of tissues of the organism or the related organism, thereby to yield the trained machine learning model. In some embodiments, each of the DNA sequences of the organism or the related organism used for training respectively contain: a gene, at least 13 kilobases upstream of a transcription start site (TSS) of the gene, and at least 13 kilobases downstream of a transcription end site (TES) of the gene. In some embodiments, each of the DNA sequences of the organism or the related organism respectively contain: a gene, at least 15 kilobases upstream of a TSS of the gene, and at least 15 kilobases downstream of a TES of the gene. In some embodiments, the input DNA sequence contains the target gene, at least 13 kilobases upstream of a TSS of the target gene, and at least 13 kilobases downstream of a TES of the target gene. In some embodiments, the input DNA sequence contains the target gene, at least 15 kilobases upstream of the TSS of the target gene, and at least 15 kilobases downstream of the TES of the target gene.

In some embodiments, the trained machine learning model includes a set of convolutional layers and a set of attention layers, in which the set of convolutional layers is configured to create embeddings from the DNA sequence that are provided to the set of attention layers, such that the trained machine learning model is capable of receiving a variable-length DNA sequence as input. In some embodiments, the relative predicted abundance of expression of the target gene across the multiple types of tissues includes relative predicted abundance of messenger ribonucleic acid (mRNA) expression of the target gene across the multiple types of tissues of the organism, or relative predicted abundance of protein expression of the target gene across the multiple types of tissues of the organism.

In some embodiments, prior to altering the portion of the plurality of nucleotides of the DNA sequence located upstream and/or downstream of the target gene, the method includes evaluating a proposed alteration by providing a second DNA sequence containing the target gene and the proposed alteration of the portion of the plurality of nucleotides of the DNA sequence located upstream and/or downstream of the target gene as a second input to the trained machine learning model, and receiving, as a second output from the trained machine learning model, a tissues of the organism. In some embodiments, altering the portion of the plurality of nucleotides of the DNA sequence includes removing at least one nucleotide, modifying at least one nucleotide, replacing at least nucleotide with one or more naturally-occurring or artificial nucleotides, inserting at least one naturally-occurring or artificial nucleotide, or any combination thereof. In some embodiments, altering the portion of the plurality of nucleotides includes altering the portion of the plurality of nucleotides using CRISPR/Cas9. In some embodiments, the multiple types of tissues of the organism include leaf tissue, embryonic tissue, anther tissue, inflorescence tissue, endosperm tissue, root tissue, or any combination thereof.

One such embodiment of a method is a method of engineering the mRNA abundance of a target gene. The method includes training a machine learning model to predict the mRNA abundance of genes in one or more tissues, cell types, growing conditions, or other variation on experimental conditions given DNA sequence as input. The method includes employing the trained machine learning model to predict the mRNA abundance of a gene of interest given a set of DNA sequence associated with the gene. The method includes determining the saliency of individual DNA nucleotides within the DNA sequence used for prediction of the gene of interest. The method includes identifying regions within the DNA sequence provided for the gene of interest with high saliency in predicting the mRNA abundance of the gene of interest. The method includes altering the sequence of high saliency regions whether via gene editing or other suitable technologies.

In some embodiments, the model is trained specifically to predict the relative, rather than absolute, mRNA abundance of genes of interest across multiple tissues, cell types, growing conditions, or other variation on experimental conditions. In some embodiments, the model is employed to predict the effect of specific DNA sequence changes in high saliency regions and only DNA-sequence changes which are predicted to produce desired changes in mRNA abundance are introduced into the DNA sequence via gene editing or other technologies. In some embodiments, saliency is calculated by comparison to observed mRNA abundance levels for the gene of interest. In some embodiments, the saliency is calculated without reference to observed mRNA abundance. In some embodiments, the model is trained using mRNA abundance, and DNA sequence data from one species or a plurality of species and is employed to engineer changes in the mRNA abundance of a species included in the training dataset.

One such embodiment of a method is a method of using a machine learning model to predict the mRNA abundance of genes given DNA sequence as input. The method includes employing one or more convolutional layer to create embeddings of input DNA sequence. The method includes providing those embeddings as input to one or more attention layers. The method includes employing the output of the attention layer or layers to predict the mRNA abundance either directly or via additional layers.

In some embodiments, the output of the attention layer or layers is employed as an input to one or more fully connected layers prior to prediction of mRNA abundance. In some embodiments, a combination of convolutional layers and pooling are employed to reduce the dimensions of the input DNA sequence. In some embodiments, averaging within embeddings produced by the attention layer or layers is used to produce a fixed number of outputs from the output layer, regardless of the length of the input DNA sequence provided. In some embodiments, the model is trained to predict features or characteristics of genes other than mRNA abundance.

Other objects, features, and advantages of the disclosure will become apparent from the foregoing figures, detailed description, and examples. It should be understood, however, that the figures, detailed description, and examples, while indicating specific embodiments of the disclosure, are given by way of illustration only and are not meant to be limiting. Additionally, it is contemplated that changes and modifications within the spirit and scope of the disclosure will become apparent to those skilled in the art from the detailed description. In further embodiments, features from specific embodiments may be combined with features from other embodiments. For example, features from one embodiment may be combined with features from any of the other embodiments. In further embodiments, additional features may be added to the specific embodiments described herein.

What is claimed is:

1. A machine learning model trained to predict a pattern of expression of a target gene contained in an input DNA sequence of an organism, the machine learning model comprising:
(i) a set of convolutional layers configured to generate embeddings of the input DNA sequence;
(ii) a positional encoding layer configured to receive the embeddings of the input DNA sequence and to generate positional information for the embeddings of the input DNA sequence;
(iii) a set of multi-headed attention layers configured to receive the embeddings of the DNA sequence and the positional information and to generate attention scores; and
(iii) a set of fully connected output layers configured to receive the attention scores and provide an output indicating the relative abundance of expression of the target gene across a plurality of different tissue types of the organism.

2. The machine learning model of claim 1, wherein, in forward operation, the machine learning model is configured to receive the input DNA sequence containing the target gene and to provide the output indicating the relative abundance of expression of the target gene across the plurality of different tissue types of the organism.

3. The machine learning model of claim 1, wherein, in reverse operation, the machine learning model is configured to receive the input DNA sequence containing the target gene and a second input indicating the relative abundance of expression of the target gene across the plurality of different tissue types of the organism, and is further configured to provide a second output comprising a respective saliency score for each of a plurality of nucleotides of the input DNA sequence located upstream and/or downstream of the target gene estimated via a gradient-based approach.

4. The machine learning model of claim 1, comprising a set of max pooling layers interposed between certain convolutional layers of the set of convolution layers, wherein the set of max pooling layers and the embeddings generated by the set of convolutional layers are configured to reduce dimensions of the input DNA sequence, such that the machine learning model is capable of receiving a variable-length input DNA sequence.

5. The machine learning model of claim 4, wherein each max pooling layer of the set of max pooling layers has a respective kernel size of 2 and a respective step size of 2.

6. The machine learning model of claim 1, wherein the set of convolutional layers comprises six convolutional layers, each convolutional layer having a respective kernel size of 15, and the six convolutional layers respectively having 1000, 500, 250, 500, 500, and 1000 feature maps.

7. The machine learning model of claim 1, wherein each convolutional layer of the set of convolutional layers is followed by a Rectified Linear Unit (RELU) activation function.

8. The machine learning model of claim 1, wherein the set of multi-headed attention layers comprises 5 multi-headed attention layers, and wherein each multi-headed attention layer comprises 1000 embedding dimensions, 8 attention heads, and a skip connection incorporating the positional information from the positional encoding layer.

9. The machine learning model of claim 1, wherein the set of fully connected output layers comprises 3 fully connected output layers respectively having sizes of 4000, 1000, and 6.

10. The machine learning model of claim 1, wherein the output contains a fixed number of values indicating the relative abundance of expression of the target gene across the plurality of different tissue types of the organism regardless of a length of the input DNA sequence.

11. The machine learning model of claim 1, wherein the output indicates the relative abundance of expression of the target gene across the plurality of different tissue types of the organism in terms of:
(i) relative abundance of messenger ribonucleic acid (mRNA) expression across the plurality of different tissue types of the organism; or
(ii) relative abundance of protein expression of the target gene across the plurality of different tissue types of the organism.

12. A method of engineering expression of a target gene of a deoxyribonucleic acid (DNA) sequence of an organism, the method comprising:
providing the DNA sequence containing the target gene as input to a trained machine learning model, and receiving, as output from the trained machine learning model, (i) relative predicted abundance of expression of the target gene across multiple types of tissues of the organism and (ii) a respective saliency score for each of a plurality of nucleotides of the DNA sequence located upstream and/or downstream of the target gene;
selecting a portion of the plurality of nucleotides of the DNA sequence located upstream and/or downstream of the target gene having high saliency in predicting abundance of expression of the target gene; and
altering the portion of the plurality of nucleotides of the DNA sequence located upstream and/or downstream of the target gene, thereby altering the abundance of expression of the target gene in one or more of the multiple types of tissues of the organism.

13. The method of claim 12, wherein, prior to providing the DNA sequence containing the target gene as input to a trained machine learning model, the method comprises:
training a machine learning model to encode relationships between (i) DNA sequences of the organism or a related organism containing genes and (ii) the abundance of expression of the genes in the multiple types of tissues of the organism or the related organism, thereby to yield the trained machine learning model.

14. The method of claim 13, wherein each of the DNA sequences of the organism or the related organism respectively contain: a gene, at least 13 kilobases upstream of a transcription start site (TSS) of the gene, and at least 13 kilobases downstream of a transcription end site (TES) of the gene.

15. The method of claim 12, wherein the DNA sequence contains the target gene, at least 13 kilobases upstream of a TSS of the target gene, and at least 13 kilobases downstream of a TES of the target gene.

16. The method of claim 12, wherein the trained machine learning model comprises a set of convolutional layers and a set of attention layers, wherein the set of convolutional layers is configured to create embeddings from the DNA sequence that are provided to the set of attention layers, such that the trained machine learning model is capable of receiving a variable-length DNA sequence as input.

17. The method of claim 12, wherein the relative predicted abundance of expression of the target gene across the multiple types of tissues comprises:
(i) relative predicted abundance of messenger ribonucleic acid (mRNA) expression of the target gene across the multiple types of tissues of the organism; or
(ii) relative predicted abundance of protein expression of the target gene across the multiple types of tissues of the organism.

18. The method of claim 12, wherein, prior to altering the portion of the plurality of nucleotides of the DNA sequence located upstream and/or downstream of the target gene, the method comprises:
evaluating a proposed alteration by providing a second DNA sequence containing the target gene and the proposed alteration of the portion of the plurality of nucleotides of the DNA sequence located upstream and/or downstream of the target gene as a second input to the trained machine learning model, and receiving, as a second output from the trained machine learning model, a second relative predicted abundance of expression of the target gene across the multiple types of tissues of the organism.

19. The method of claim 12, wherein altering the portion of the plurality of nucleotides comprises altering the portion of the plurality of nucleotides using CRISPR/Cas9.

20. The method of claim 12, wherein the multiple types of tissues of the organism comprise leaf tissue, embryonic tissue, anther tissue, inflorescence tissue, endosperm tissue, root tissue, or any combination thereof.
